(19) United States
(12) Patent Application Publication
(10) Pub. No.: US 2006/0149386 A1
(43) Pub. Date: Jul. 6, 2006

(54) Joint prostheses

(76) Inventors: Michael Thomas Clarke, Cambridge (GB); Paul Tee Hui Lee, Leicester (GB)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 10/495,255

(22) PCT Filed: Nov. 7, 2002
(86) PCT No.: PCT/GB02/05042

§ 371 Date: Jan. 27, 2005

(30) Foreign Application Priority Data
Nov. 7, 2001 (GB) 0126704.6

(51) Int. Cl. A61F 2/30

(52) U.S. Cl. 623/18.12

(57) ABSTRACT

A component (1) of a prosthetic joint comprising a portion to secure the component to a bone and a bearing portion having a bearing surface (3) wherein one more reservoir (6) are situated behind the bearing surface (3), one or more magnet assemblies (5) are associated with the or each reservoir (6) and one or more passages (7) are provided extending between a surface of the component and the or each reservoir (6), and wherein either the bearing surface (3) includes magnetic material or a material that in use has a magnetic surface or the component (1) is adapted for use with a further joint component (2) which has a bearing surface (4) that includes magnetic material or a material that in use has a magnetic surface.

The present invention relates to replacement joints (joint prostheses) and in particular to improvements to the longevity of such joints.

Total replacement of joints, such as hip joints, is considered as an immensely successful procedure. Despite this, the major limiting factor regarding the longevity of this type of surgery is the process of osteolysis (bone resorption) and aseptic loosening which ultimately leads to implant failure and the need for revision surgery. This phenomenon is attributed by most researchers in the field as being directly related to the production of wear particles from the bearing surface, resulting in wear debris thought to play a role in a form of immune cell reaction at the prosthesis/bone interface, causing bone resorption and implant loosening. The revision surgery needed as a result of loosening incurs a high degree of morbidity for the patient and can be complex and costly. Accordingly, the goal of many research efforts over the past 30 years in this field has been directed towards prevention of these problems.

Published research has suggested that by minimising wear debris production, the resulting bone resorption is minimised or even prevented. This philosophy has resulted in the widespread clinical use of bearing designs that avoid ultra high molecular weight polyethylene (UHMWPE), the main cause of wear related debris in currently implanted prostheses. Typical examples of new bearing designs in routine clinical use include high hardness metal-on-metal and ceramic-on-ceramic bearings. Animal and clinical investigations have also examined surface modified coatings such as titanium nitride and diamond-like carbon. Despite much research, however, no bearing surface has been shown to be ideal.

Metal-on-metal bearings are typically manufactured from cobalt-chromium-molybdenum alloy (CoCrMo; e.g. ASTM F75-98, F799-99 or F1537-00). This bearing surface produces 20-100 times less volumetric wear (1-2 mm³/year) than the standard metal or ceramic on UHMWPE. Despite this reduction in volumetric wear, the particles produced by metal-on-metal bearings are much smaller in size (down to 10 nm) than UHMWPE particles resulting in larger numbers per unit volume. This is important as the large numbers of small particles produced has the potential for exacerbating the inflammatory response rather than reducing it. Further, reports of significantly raised serum cobalt and chromium levels in patients having received these implants has raised concern about their safety. These ions have been linked with systemic disease including chromosomal damage and cancer.

Ceramic-on-ceramic bearings, mainly produced from alumina or zirconia in various combinations, can also provide a low wear bearing surface with volumetric wear rates similar to or slightly better than that of metal-on-metal. There are, however, several major drawbacks to ceramic implants. Their hardness and brittleness makes them difficult and expensive to manufacture as well as predisposing them to fracture after implantation. This fragility also requires the surgeon to insert the prosthesis with a very exacting technique making the surgery more demanding.

Surface engineering techniques, such as thin surface coatings of titanium nitride and diamond-like carbon have yet to be proven useful. Initial simulator and implant tests revealed their weakness to delamination from the underlying substrate and subsequent failure.

With the above in mind, some have acknowledged that the production of wear debris cannot be prevented, only minimised. These researchers have developed methods to prevent wear debris from reaching the prosthesis/bone interface where the end-result of bone resorption is effected. A number of inventions have included ‘encapsulating’ hip arthroplasties where the bearing is surrounded by a semi- or non-permeable membrane that traps debris. Others have attempted to attach semi-permeable (e.g. Gore-Tex) membranes around the superficial joint interfaces of the prosthesis and bone in order to prevent wear debris accessing the deeper interfaces. These devices have not met with any degree of acceptance as they are cumbersome and require extra steps during surgery that make the procedure more difficult.

The aim of the present invention is to provide a satisfactory method of preventing wear debris from reaching the prosthesis/bone interface and biological tissue that addresses the problems encountered with known methods.

According to a first embodiment the present invention provides a component of a prosthetic joint comprising a portion to secure the component to a bone, a bearing portion having a bearing surface wherein one or more reservoirs are situated behind the bearing surface, one or more magnet assemblies are associated with the or each reservoir and one or more passages are provided extending between a surface of the component and the or each reservoir, said component being adapted for use with a further joint component which has a bearing surface that includes magnetic material or a material that in use has a magnetic surface.

According to an alternative first embodiment the present invention provides a component of a prosthetic joint comprising a portion to secure the component to a bone, a bearing portion having a bearing surface wherein one or more reservoirs are situated behind the bearing surface, one or more magnet assemblies are associated with the or each reservoir and one or more passages are provided extending between a surface of the component and the or each reservoir, wherein the bearing surface includes magnetic material or a material that in use has a magnetic surface.

The bearing surface may completely, or substantially completely, be magnetic material or material that in use has a magnetic surface. Alternatively, the bearing surface may only partially be magnetic material or material that in use has a magnetic surface.

The portion to secure the component to a bone is preferably a backing portion, more preferably a metal backing portion. Most preferably the backing portion is made from non-magnetic metals and alloys such as titanium, titanium alloys (e.g. ASTM F1472-99, F1108-97a, F1295-97a, F1713-96), tantalum, CoCrMo (e.g. ASTM F75-98, F799-99 or F1537-00) and cobalt-nickel-chromium-molybdenum (CoNiCrMo; e.g. ASTM F1058-97, F562-00, F563-95, F961-96). Alternatively, the portion to secure the component to a bone may be integrated in the bearing portion.

The backing portion may suitably be modified on the outer surface by, for example, roughening or coating to enhance its immediate or subsequent fixation to bone. Examples of suitable coatings include roughened or porous CoCrMo, titanium, titanium alloy, tantalum, hydroxyapatite and combinations thereof. These coatings may be applied by suitable methods known in the art, such as thermal spraying or sintering. Such external coatings may also function to enclose the or each magnetic assembly within the joint component to prevent dislodgement, in particular during insertion or use.

The bearing portion may be part of a composite front section, which may suitably be mass-produced in a factory. The composite front section suitably comprises a non-magnetic shell that contains the or each reservoir and the or each magnet assembly associated with the or each reservoir, and the bearing portion having a bearing surface, with the bearing portion being located on the inner surface of the shell. Preferably the composite front section further comprises an additional layer, preferably a non-magnetic layer, for example, a polymer layer such as an ultra high molecular weight polyethylene (UHMWPE) layer, on the outer surface of the shell. The composite front section can be securely attached, for example by being impacted, to the backing portion using standard methods to produce the component.

Alternatively, the bearing portion may be an individual section that can be attached directly to the portion to secure the component to a bone. The outer surface of the bearing portion may be coated with an appropriate material that encloses the or each magnet assembly within the component to prevent dislodgement of the or each magnet assembly during insertion or use. Further, the coating improves the ability of the bearing portion to be attached to the portion to secure the component to bone.

The bearing portion may also be an individual section that integrates the portion to secure the component to a bone. The outer surface of said bearing portion may be coated with an appropriate material that encloses the or each magnet assembly within the component to prevent dislodgement of the or each magnet assembly during insertion or use. This coating may also form part or all of the portion to secure the component to the bone.

The bearing portion may be constructed wholly or partially from a magnetic material. More preferably the bearing surface of the bearing portion is wholly or partially coated with a magnetic material. In one arrangement the magnetic material coats only the part of the bearing surface that generates the majority of the debris.

The magnetic material may be made from magnetically hard or semi-hard material but is preferably made from magnetically soft material with low coercivity such that it does not become permanently magnetised by the effects of bearing friction or strong external magnetic fields, such as used in Magnetic Resonance Imaging (MRI) processes. Preferably the materials have a coercivity of less than 12 Oersteds, more preferably less than 6 Oersteds and most preferably as close to zero as possible. The material ideally has high saturation magnetic flux density (at least 0.3 Tesla, preferably greater than 1.0 Tesla and more preferably greater than 2.0 Tesla) and high relative permeability (at least 50, preferably greater than 100 and more preferably greater than 1000) such that wear debris created in use around the joint component is pulled towards the or each magnet assembly.

Suitable magnetic materials include metal alloys, metal oxides, intermetallic compounds, ceramics, amorphous metal alloys and combinations thereof including metal matrix composites.

Metal alloys suitable for use include:

1. Cobalt based alloys;

2. Nickel based alloys; and

3. Iron based alloys including magnetic steels.

In general, alloys that satisfy the following conditions (in addition to possessing optimal wear and corrosion resistance for long-term implant bearing use) in terms of % weight are preferred.

60≦(Co+Fe+Ni)≦95

0≦(Mo+Cr)≦35

0≦Co≦90, preferably from 50 to 70

0≦Fe≦90, preferably from 0 to 20

0≦Ni≦90, preferably from 0 to 40

0≦Cr≦30, preferably from 5 to 15

0≦Mo≦20, preferably from 2 to 10

0≦C≦10, preferably from 0 to 6;

with any balance being made up from: Mn, Ti, Ta, Si, Al, S, P, N, V, B, Nb, Cu, Hf, Zr, W, preferably at a level of ≦15.

For example

Co 62, Ni 13, Cr 12, Mo 4, Fe 9, C<0.2
Co 68, Cr 14, Ni 9, Fe 5, Mo 4, C<0.2
Co 85, Cr 15
Fe 82, Cr 17, C 1
Fe 67, Cr 29, Mo 4
Fe 67, Cr 22, Ni 6, Mo 3.
Ni 51, Fe 48, Mn 0.5, Si 0.35

Suitable ceramics include oxides and nitrides of nickel, iron, cobalt and manganese.

e.g.

CrO₂
Fe₃O₄
CoFe₃O₄
NiO
Fe_{(2 to 4)}N

Ceramic materials having magnetic properties may be preferable to alloys for use as the magnetic material. The particulate wear debris from many ceramics is highly corrosion resistant and may retain its magnetic properties in the long term. This is in contrast to metal alloys, where the bearing surface and particulate wear debris produced may experience a leeching out of the more soluble ions with time (e.g. cobalt ions with CoCrMo alloys). This potentially can be detrimental to the magnetic properties of the bearing surface and particulate wear debris.

Amorphous metal alloys suitable for use in the present invention may have compositions similar to the metal alloys above but with high silicon and/or boron content (e.g. Co 82, Si 8.6, Fe 4.45, B 3.15, Ni 1.63).

The coating of magnetic material on the bearing surface may be formed by applying a layer of magnetic material to the bearing surface using coating techniques. Alternatively, a coating can be produced by treating the bearing surface so as to chemically alter the material at the surface and thus cause the formation of a layer of magnetic material. Any known coating techniques and treatment methods for producing a coating may be used. Without limitation, suitable methods for achieving a magnetic coating on the bearing surface may include welding, diffusion bonding, thermal spraying, ion implantation, plasma vapour deposition, cathode vapour deposition, oxidation, nitriding (by any of the many methods available), hot isostatic pressing, sintering and laser cladding.

Alternatively, the bearing portion may be wholly or partially constructed from a material that in use has a magnetic surface or the bearing surface may be wholly or partially coated with a material that in use has a magnetic surface. In one arrangement the material that in use has a magnetic surface coats only the part of the bearing surface that generates the majority of the debris.

Generally, the material may be non-magnetic under normal circumstances but when used in the bearing portion or as a coating on the bearing surface it becomes partially or completely magnetic, with at least the exposed surface of the material being magnetic. In particular, it is preferred that in use at least the external surface of the material is a magnetic material having properties as described above. For example, the bearing portion may be constructed from or the bearing surface coated with an alloy that is generally non-magnetic but during use becomes magnetic at at least the surface due to strain induced hardening. Alternatively, the bearing portion may be constructed from or the bearing surface coated with an alloy that is generally non-magnetic but that in use develops thin layers of magnetic oxides at the surface due to corrosion.

Wholly or partially coating the bearing surface, preferably with a magnetic material or alternatively with a material that in use has a magnetic surface, is preferred to forming the bearing portion from a magnetic material or a material that in use has a magnetic surface. This is because in coating the bearing surface a smaller amount of magnetic material is used, which reduces the forces exerted by high external magnetic fields such as with MRI scanners. Additionally, if the bearing surface is coated with magnetic material, or if the coating is applied only to the part of the bearing surface that generates the majority of the debris (for example, the polar region of a ball and socket joint as opposed to the equatorial region), rather than the magnetic material comprising a substantial part of the bearing portion, the magnetic field from the or each magnet assembly used to attract particles can penetrate more efficiently the area or areas where the particulate debris is formed or passes. This allows the use of lower strength, smaller and more cost-effective magnets and magnet assemblies. This, in turn, minimises the forces exerted on the prosthesis by external magnetic fields. In addition, the effect of the or each magnetic assembly on surrounding biological tissue is reduced, and therefore the need for magnetic shielding of the or each magnetic assembly when a biological effect is undesirable is reduced.

The or each magnet assembly may comprise one or more permanent magnets. Clearly, any magnet that has sufficient power to attract particles may be used. Examples of suitable magnets include samarium-cobalt (SmCo) or neodymium-iron-boron (NdFeB) magnets. The magnets may be coated, for example, with a corrosion resistant material such as a corrosion resistant polymer or a corrosion resistant metal such as chromium or nickel. The aforementioned types of magnet have high coercivity and high maximum energy products (BHmax) and in addition are resistant to radiation, shock and time related demagnetisation.

The magnetic field produced by the or each permanent magnet assembly is preferably externally shielded. This allows the effect of the field on surrounding biological tissue to be minimised. Further, such shielding also reduces the impact of external magnetic fields (for example MRI scanners) on the magnet itself, and can therefore help prevent demagnetisation. Such shielding can suitably be achieved by methods known in the art such as the use of one or more magnetic materials situated at suitable intervals around the magnet. The shielding material and the or each magnet assembly may be coated together as one or more units with a corrosion resistant material as described above. The magnetic permeability of the magnetic material on the bearing surface may also be chosen such that this material acts as a shield, or a layer of a suitable shielding material may be comprised within the component.

The material of the backing portion may alternatively, or additionally, be chosen such that the backing portion acts as a shield. For example, the backing portion may contain one or more magnetic shielding layers externally or, preferably, internally.

The extent of shielding can be limited or removed to allow application of a magnetic field of an appropriate strength to the surrounding biological tissue, giving rise to an appropriate therapeutic effect. For example, bone growth around the prosthesis may be enhanced by the application of a therapeutic magnetic field. This could enhance the subsequent fixation of the prosthesis to bone and minimise the possibility of dislodgement or loosening.

The entrances to the passages extending from a surface of the components to the or each reservoir may be located at any point on the surface of the components. Preferably the entrances to the passages are located on a surface which in use is not adjacent to biological tissue. In a preferred embodiment the entrances to the passages are located on or near the bearing surface. In particular, they may be arranged around the circumference of the bearing surface of the component, or they may be located on a part of the bearing surface distinct from the circumference. When the entrances to the passages are arranged around the circumference of the bearing surface of the component, they may be arranged around the entire circumference or may be arranged around only part of the circumference, for example around a quarter or half section of the circumference. In one arrangement, the entrances to the passages are located only on the area of the bearing surface that, in use, is not load bearing. Preferably there are several passages extending from a surface of the components to the or each reservoir, for example 4 or more, preferably 10 or more, more preferably 20 or more.

Clearly the passages and reservoirs should be of sufficient size so as to be able to contain particles of the magnetic material. The total capacity of all of the passages and reservoirs should be sufficient to contain the maximum total volume of wear debris particles that could be expected to be produced during the life of the patient. It is preferred that the total capacity of all the passages and reservoirs is significantly greater than the volume of particles that could be expected to be produced during the life of the patient, so that the passages and reservoirs are unlikely to become clogged with material. For this reason it is also preferred that the size of the entrance to each passage on the surface of the socket is at least several times the size of the largest particles of magnetic material. The particles of magnetic material may be as small as 5 to 10 nm. Depending on the nature of the magnetic material, larger particles of up to 10 μm may be produced and occasionally very large particles of up to 500 μm may be produced. It is clearly desirable that the passages and reservoirs do not compromise the bearing surface or compromise structural support for the bearing surface. Accordingly, the passages may, for example, have approximately circular entrances of from 0.5 to 5 mm in diameter. The depth of the passages may be, for example, from 0.5 to 5 mm.

It is further preferred that the passages are designed such that once the particles have entered the passage they are unlikely to exit it under normal circumstances. For example, each passage may have an entrance portion and an end portion, with the entrance portion being wider than the end portion.

According to a second embodiment the present invention provides a prosthetic joint suitable for replacing an existing joint comprising two components dimensionally adapted to articulate with each other wherein one or both components are provided in accordance with one of the first embodiments of the invention as described above and wherein one or both joint components have a bearing surface including a magnetic material or a material that in use has a magnetic surface.

Preferably, only one component of the joint is provided in accordance with one of the first embodiments described above. The other component of the joint may be made from any suitable material such as a ceramic or CoCrMo alloy. One or both of the components may have a bearing surface including a magnetic material or a material that in use has a magnetic surface.

Alternatively, both components of the joint may be provided in accordance with one of the first embodiments as described above. The bearing surface of one or both of these components may include a magnetic material or a material that in use has a magnetic surface.

The joint component and resultant joint of the present invention are highly advantageous, as owing to the nature of the material that forms the bearing surface of one or both portions of the joint, the wear debris created in use is at least partially magnetic. The, or at least part of the, wear debris is therefore attracted by the magnet assembly into the reservoir. The amount of debris reaching the prosthesis/bone interface and biological tissues is therefore reduced or eliminated, alleviating the problems associated with wear debris reaching the interface and with metal ions reaching biological tissue. A further advantage of the joint component and resultant joint of the present invention is that they may be implanted into a patient using standard surgical techniques for implanting prostheses. Also, the risk of joint dislocation, subluxation or micro-separation may be reduced with the joint of the present invention, due to the magnetic pull on one component by the magnet assembly in the other component.

In a further embodiment the present invention provides a system suitable for replacing a hip joint, which comprises a head and a socket dimensionally adapted to articulate with the head, wherein the socket is provided in accordance with one of the first embodiments of the invention, the head has a portion for securing the head to a bone and one or both of the bearing surface of the socket and the surface of the head substantially comprise magnetic material or material that in use has a magnetic surface.

Alternatively, the system may comprise a head and a socket dimensionally adapted to articulate with the head, wherein the head is provided in accordance with one of the first embodiments of the invention, the socket has a portion for securing the socket to a bone and a bearing surface, and one or both of the bearing surface of the socket and the surface of the head substantially comprise magnetic material or a material that in use has a magnetic surface.

The head and socket may each be of any shape, provided that they can articulate together to mimic the movement of the joint to be replaced. Preferably the head is substantially sphere shaped and the socket is of a corresponding shape that can engage with the curved sphere surface. In particular, the socket may have a corresponding cup (hollow hemisphere) shape or may be in the shape of a part of such a hemisphere. The size of the head and the socket is chosen depending on their intended use.

The head and the socket may each be made entirely or in part from magnetic material or material that in use has a magnetic surface, with each comprising the same or different materials. Preferably both the surface of the head and the surface of the socket that articulates with the head partially or substantially comprise magnetic material. In one embodiment one or both of these surfaces is entirely magnetic material. In particular the head may be coated with magnetic material and/or the socket may be coated with magnetic material in the area that articulates with the head. In a preferred embodiment the head and the socket are both made substantially from non-magnetic material, but contain magnetic material in the area where the head articulates with the socket and the area where the socket articulates with the head.

Magnetic materials suitable for use in the system are those described above in relation to the first embodiment of the invention. It is preferred that the magnetic material is an alloy or ceramic, for example a cobalt, nickel or iron based alloy or ceramic with suitable corrosion and wear resistance for implant bearing use.

Preferably the head and the socket do not entirely comprise magnetic material or material that in use has a magnetic surface. In this case any non-magnetic materials suitable for use in implants may be used to form the remainder of each component. In particular, non-magnetic metals, alloys and ceramics, for example titanium, titanium alloys (e.g. ASTM F1472-99, F1108-97a, F1295-97a, F1713-96), tantalum, CoCrMo (e.g. ASTM F75-98, F799-99 or F1537-00) and cobalt-nickel-chromium-molybdenum (CoNiCrMo; e.g. ASTM F1058-97, F562-00, F563-95, F961-96), zirconia and alumina, may be mentioned. The head and the socket may each comprise one or more different non-magnetic materials.

Preferably the socket comprises a backing portion and a composite front section, which may suitably be mass-produced in a factory. The composite front section can be fixed (e.g. impacted) into the backing portion to produce the socket. Alternatively, the socket may comprise a backing section which may be used to attach the socket to bone and a bearing section, or may be an individual bearing section which has an integrated portion for attaching the socket to bone.

Preferably the socket or the head has one magnet assembly and one associated reservoir. The magnet assembly and the reservoir are preferably circumferential, for example they may extend around the opening of the cup shaped socket or around the circumference of the head.

Preferably the head comprises a core section and one or more outer layers. The outermost layer of the head, which forms the bearing surface, preferably includes a magnetic material. The core section preferably comprises a non-magnetic material such as titanium, titanium alloy, CoCrMo, CoNiCrMo, stainless steel or non-magnetic ceramic.

The permanent magnet assembly in the socket or head is preferably circumferential; for example it may be loop shaped. It is preferred that the magnet assembly is located inside the socket or head such that it is coplanar with at least one point of the bearing surface of the head or the socket. More preferably the magnet assembly is located such that it is coplanar with at least two points of the bearing surface of the head or the socket.

Preferably, the magnetic assembly provides suitable field strength and is located such that a magnetic field is produced over the entire area where the head and socket articulate. The magnetic field may also extend to at least some of the surrounding area as well as the area of direct articulation. Alternatively, the magnetic field may extend only over a specific localised area of the joint system, for example the area around the front edge of the socket. Clearly it is preferable that the field is such that all particles of magnetic material that come away from the surfaces during the life of the implant are within the magnetic field at some point to the extent that allows them to be attracted towards the magnetic assembly and reside in the reservoirs.

Both the head and the socket comprise portions for securing to a bone that allow the head and the socket to each be attached to appropriate bones in the body during surgery.

Although the joint replacement system has been described with reference to the replacement of a hip joint, it will be understood that the system may also be used to replace other joints, such as knee joints or shoulder joints.

Further provided is the use of a joint component or prosthetic joint according to the present invention to reduce the amount of wear debris reaching the prosthesis/bone interface in a prosthetic joint.

In addition there is provided the use of a joint component or prosthetic joint according to the present invention to reduce or prevent osteolysis.

In addition there is provided the use of a joint component or prosthetic joint according to the present invention to reduce or prevent aseptic loosening.

The above uses of the joint component or prosthetic joint of the present invention reduce, or eliminate, the need for revision surgery which has a high associated morbidity rate and arises due to failure of a prosthetic joint through osteolysis or aseptic loosening.

In addition there is provided the use of a joint component or prosthetic joint according to the present invention to reduce the level of circulating and tissue metal ions and wear particles. The metal ions may be, for example, chromium, nickel or cobalt. The presence of circulating and tissue metal ions has been linked with systemic disease such as chromosomal damage and cancer.

Particular embodiments of the invention are further described with reference to the accompanying drawings, which are not intended to limit the scope of the invention.

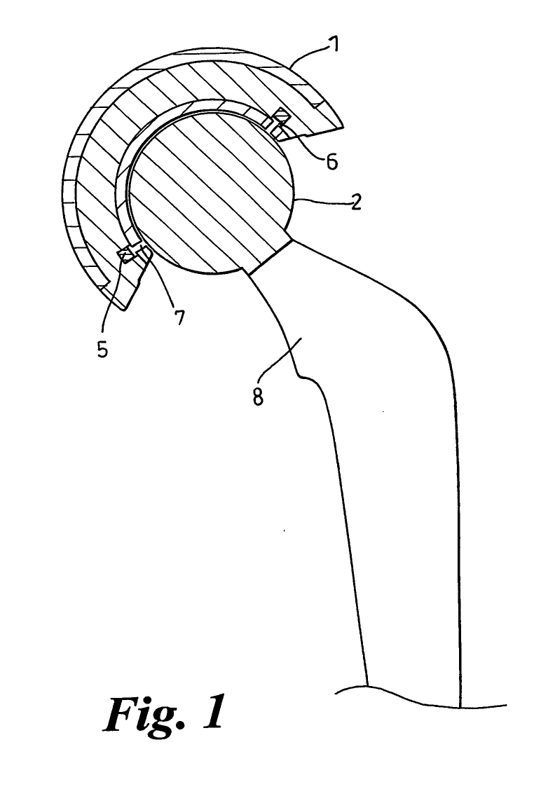

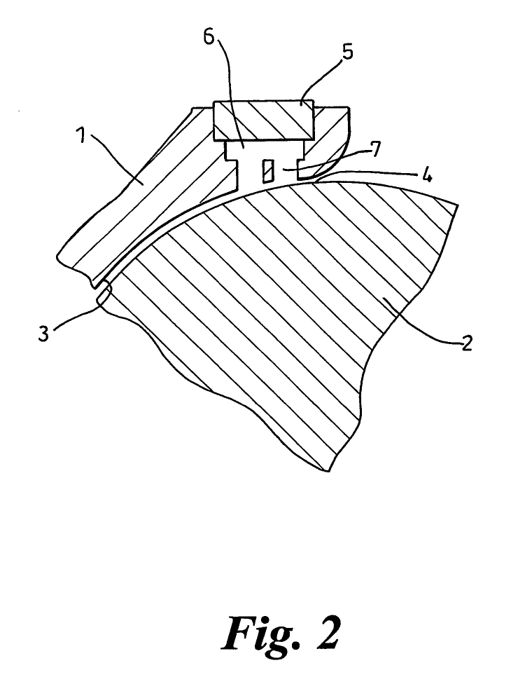

FIG. 1 is a diagrammatic representation of a system of the present invention as applied to a hip replacement;

FIG. 2 is a close up view of a system of FIG. 1 of the present invention showing the articulating surfaces of the head and the socket;

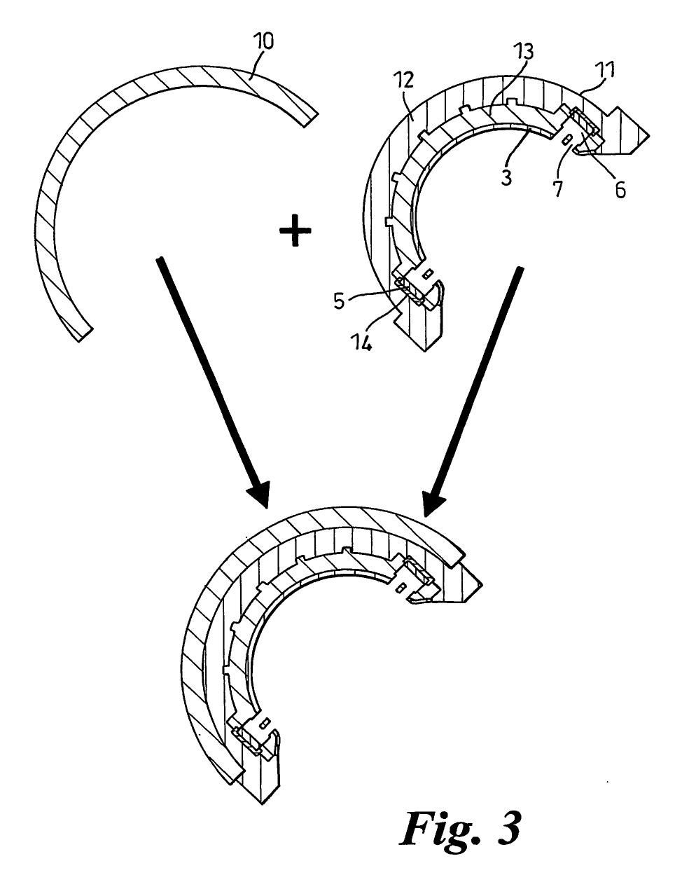

FIG. 3 is a diagrammatic representation showing the detailed composition of a system according to FIG. 1 of the present invention;

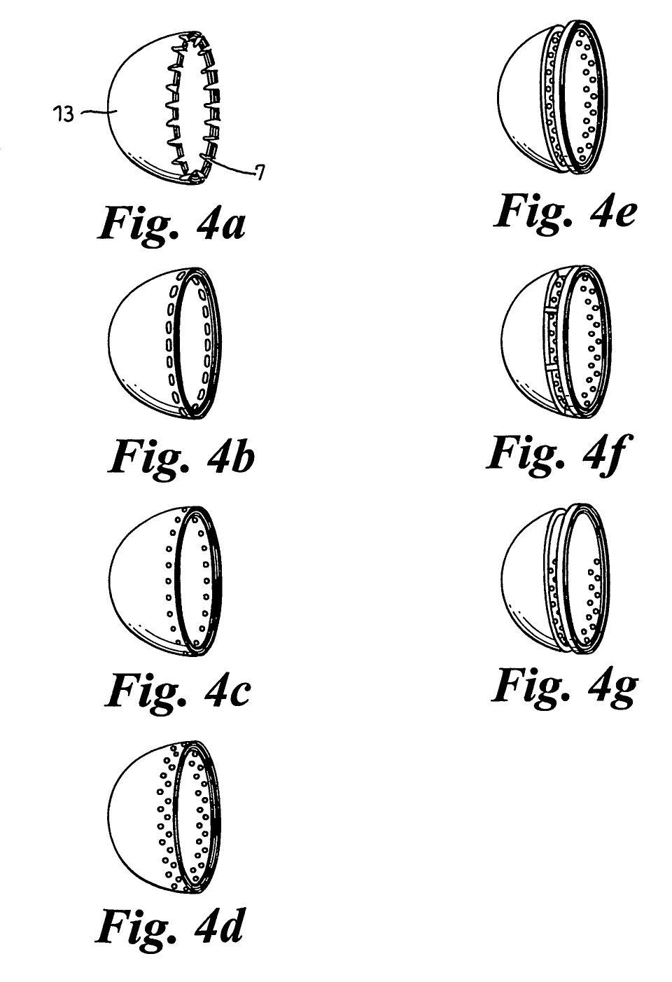

FIGS. 4a-4g show examples of shells suitable for use in a socket component of FIG. 1 of the present invention;

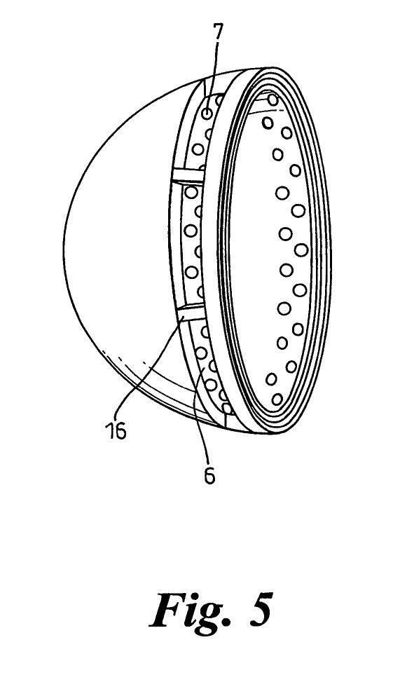

FIG. 5 is a diagrammatic representation showing the detailed composition of a shell according to FIG. 4f;

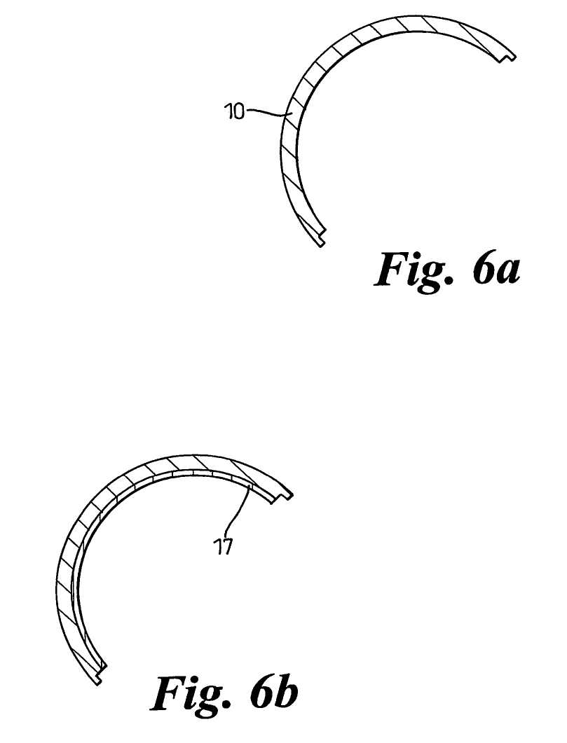

FIGS. 6a and 6b show examples of a backing suitable for use in a socket component of FIG. 3;

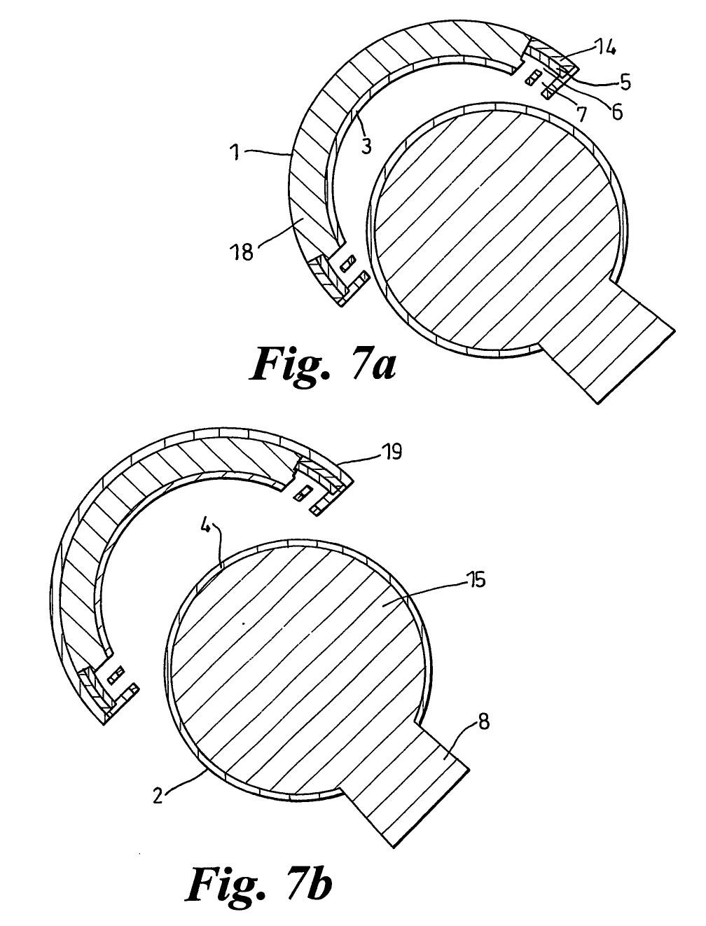

FIGS. 7a and 7b are diagrammatic representations showing the detailed composition of examples of a second system according to FIG. 1 of the present invention;

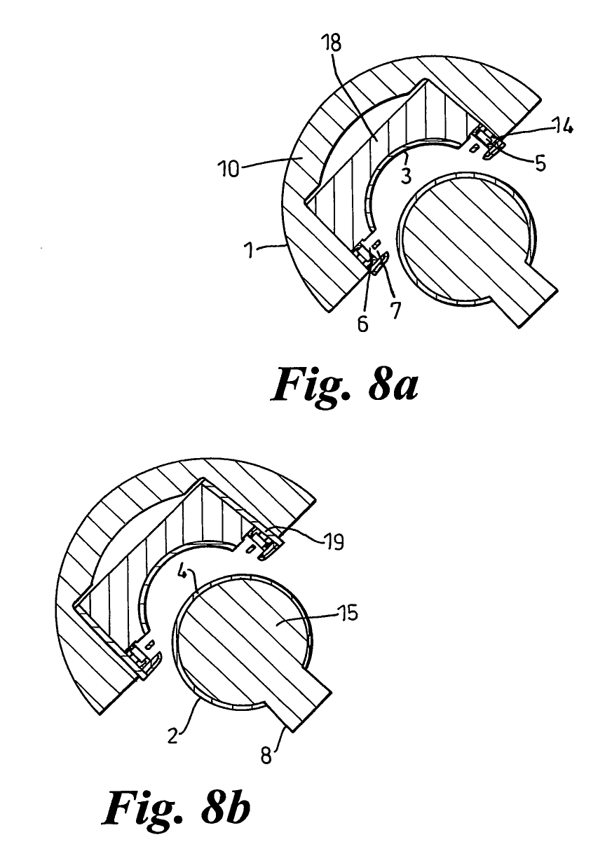

FIGS. 8a and 8b show the detailed composition of examples of a third system according to FIG. 1 of the present invention;

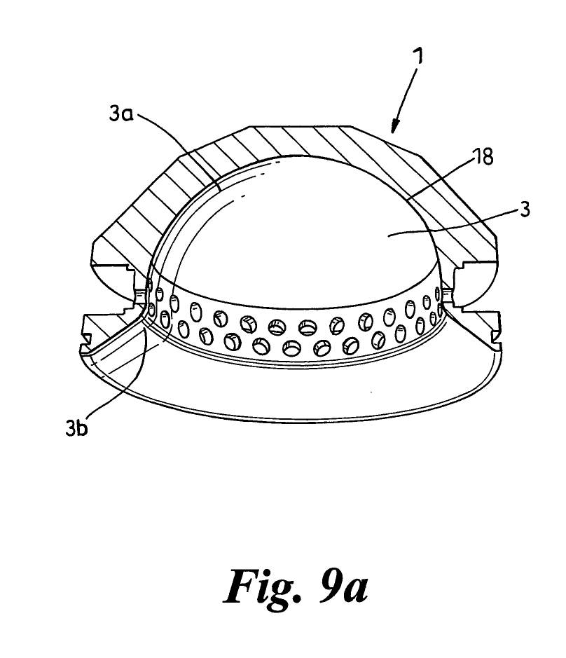

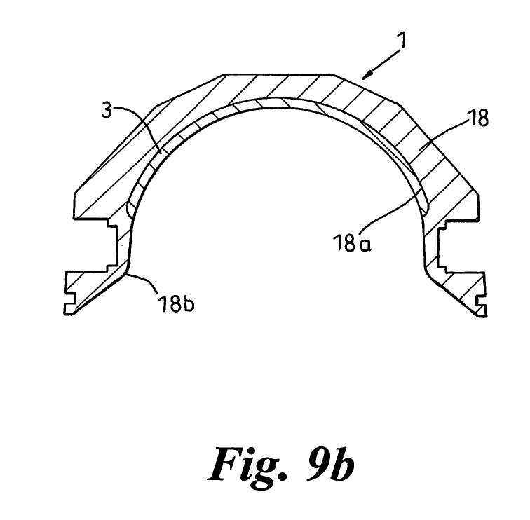

FIGS. 9a and 9b show a partial magnetic coating on a socket of the present invention in perspective view and in cross-section; and

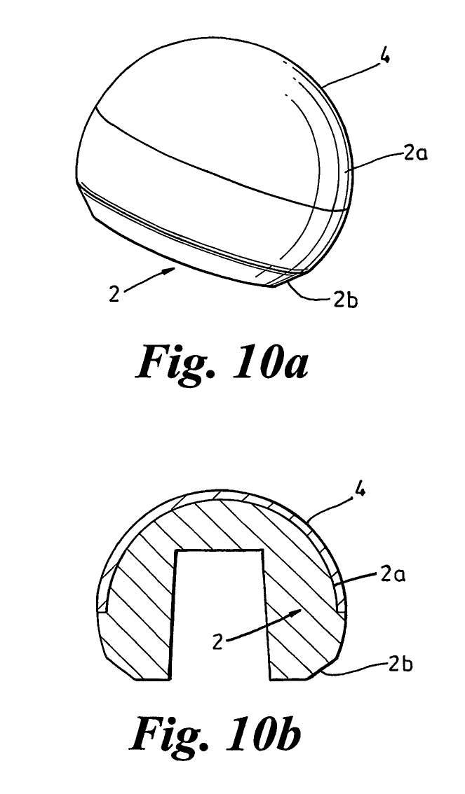

FIGS. 10a and 10b show a partial magnetic coating on a head component of the present invention in perspective view and in cross-section.

FIGS. 1 and 2 show an embodiment of a prosthetic joint according to the invention. The joint comprises a socket component 1 and a head component 2 that articulate together. The bearing surface 3 of the socket component and the bearing surface 4 of the head component each include a magnetic material. The socket component 1 includes a circumferential magnet system 5 associated with a reservoir 6 that has passages 7 leading to the bearing surface. The head component 2 has a securing means 8 that may be attached to a bone.

In use the particles of bearing surfaces 3 and 4 that come away from the surface due to wear are attracted to the magnet 5. This attraction will cause them to move towards one of the passages 7 and then into the reservoir 6 where they will remain due to their attraction to the magnet. Accordingly, the particles are prevented from reaching the bone/prosthesis interface and thus a major cause of osteolysis (bone resorption) is removed.

FIG. 3 shows, in detail, an embodiment of the socket component of a prosthetic joint according to FIG. 1 of the invention. The socket component 1 comprises a backing portion 10 and a composite front portion 11. The composite front portion 11 comprises a polymer layer 12, positioned in use against the backing portion. A non-magnetic shell 13 is positioned between the polymer layer 12 and the bearing surface 3. A circumferential magnet system 5 is situated in the non-magnetic shell 13 and has a shielding layer 14 to shield the magnet system from biological tissue. The magnet system 5 is associated with a reservoir 6 that has passages 7 leading to the bearing surface.

FIGS. 4a-4g show examples of a shell 13 for use in a socket component 1 according to the invention. Each shell 13 comprises a number of passages 7. As can be seen, the passages may vary in size, shape and number. In FIG. 4a cut-outs around the edge of the shell are provided, in FIG. 4b elongate holes are provided in the shell and in FIGS. 4c, 4d, 4e and 4f small circular holes are provided. In FIG. 4g, small circular holes around part of the circumference are provided.

FIG. 5 shows in detail the construction of the shell of FIG. 4f. The inner surface is reinforced with radial supports 16 that are situated at intervals within the reservoir 6, which is linked by passages 7 to the inner surface.

FIGS. 6a and b show examples of a backing portion 10 for use in a socket component 1 according to the invention. FIG. 6a shows a basic backing portion made from metal. In FIG. 6b the backing portion further comprises a layer of magnetic shielding material 17 on the inner surface, which assists in the shielding of biological tissue from the magnetic field of the magnet assembly.

FIGS. 7a and b show, in detail, a second embodiment of the components of a prosthetic joint according to FIG. 1. The socket 1 is a one-piece component, comprising a bearing portion 18 with a bearing surface 3. A circumferential magnet system 5 is situated in bearing portion 18 and has a shielding layer 14 to shield the magnet system. The magnet system 5 is associated with a reservoir 6 that has passages 7 leading to the bearing surface. In FIG. 7b the bearing portion 18 further has an external coating 19 that provides for appropriate securing of the bearing portion 18 to bone and that encloses the magnet system 5, preventing dislodgement during insertion or use.

The head component 2 comprises a non-magnetic core 15, a bearing surface 4 and a securing means 8.

FIGS. 8a and b show, in detail, a third embodiment of the components of a prosthetic joint according to FIG. 1. The socket 1 is a two-piece component, comprising a bearing portion 18 with a bearing surface 3 and a backing portion 10 that are impacted together. A circumferential magnet system 5 is situated in the bearing portion 18 and has a shielding layer 14 that shields the magnet system. The magnet system 5 is associated with a reservoir 6 that has passages 7 leading to the bearing surface 3. In FIG. 8b the bearing portion 18 has an external coating 19 that can appropriately secure bearing portion 18 to the backing portion 10 and that encloses the magnet system 5, preventing dislodgement during insertion or use.

The head component 2 comprises a non-magnetic core 15, a bearing surface 4 and a securing means 8.

FIGS. 9a and b show a further embodiment of the invention wherein the magnetic coating forming the bearing surface 3 is applied to only a part of the bearing portion 18 of the prosthetic joint socket 1 of the present invention. In FIGS. 9a and 9b the magnetic coating is applied only to the polar region 18a of the bearing portion 18 of the socket 1 rather than the polar 18a and equatorial 18b regions.

FIGS. 10a and b show a further embodiment of the invention wherein the magnetic coating forming the bearing surface 4 is applied to only a part of the prosthetic head component 2 of the present invention. In FIGS. 10a and 10b the magnetic coating is applied only to the polar region 2a of the head component rather than the polar region 2a and the equatorial region 2b.

1. A component of a prosthetic joint comprising:
a portion to secure the component to a bone;
a bearing portion having a bearing surface wherein at least one reservoir is situated behind the bearing surface
at least one magnet assembly associated with the reservoir; and
at least one passage extending between a surface of the component and the reservoir, said component being adapted for use with a further joint component which has a bearing surface that includes at least one of a magnetic material and a material that in use has a magnetic surface.

2. A component of a prosthetic joint comprising:
a portion to secure the component to a bone;
a bearing portion having a bearing surface wherein at least one reservoir is situated behind the bearing surface, and wherein the bearing surface includes at least one of a magnetic material and a material that in use has a magnetic surface;
at least one magnet assembly associated with the reservoir; and
at least one passage extending between a surface of the component and the reservoir.

3. A component according to claim 1 wherein the portion to secure the component to a bone is a backing portion made from at least one of a non-magnetic metal, a non-magnetic alloy, titanium, a titanium alloy, tantalum, CoCrMo, and cobalt-nickel-chromium-molybdenum.

4-6. (canceled)

7. A component according to claim 1 wherein the backing portion has at least one of an outer surface having one of a roughened surface and a coating thereon and one of a roughened and porous coating of at least one of CoCrMo, titanium, titanium alloy, tantalum, and hydroxyapatite.

8. (canceled)

9. A component according to claim 1 wherein the bearing portion is part of a composite front section comprising:
a non-magnetic shell that contains the reservoir and the magnet assembly associated with the reservoir;
the bearing portion, wherein the bearing portion is located on the inner surface of the shell; and
an additional non-magnetic layer on an outer surface of the shell.

10-11. (canceled)

12. A component according to claim 1 wherein the bearing portion is at least one of an individual section attachable directly to the portion to secure the component to a bone, an individual section integrated with the portion to secure the component to a bone, one of wholly and partially constructed from a magnetic material, and one of wholly and partially coated with a magnetic material.

13. (canceled)

14. A component according to claim 12 wherein an outer surface of the bearing portion is coated with a material that encloses the magnet assembly within the component to prevent dislodgement of the magnet assembly during at least one of insertion and use.

15-16. (canceled)

17. A component according to claim 12 wherein the magnetic material is at least one of a magnetically soft material with low coercivity and a magnetic material having a high saturation magnetic flux density and high relative permeability such that wear debris created in use around the joint component is pulled towards the magnet assembly.

18. (canceled)

19. A component according to claim 12 wherein the magnetic material is selected from a group consisting of metal matrix composites, metal alloys, a metal alloy selected from cobalt based alloys, nickel based alloys, magnetic steels, and iron based alloys, metal oxides, a metal oxide selected from oxides of nickel, iron, cobalt, and manganese, intermetallic compounds, ceramics, a ceramic selected from oxides and nitrides of nickel, iron, cobalt, and manganese, amorphous metal alloys, an amorphous metal alloy selected from cobalt, nickel, and iron based amorphous alloys with at least one of a high silicon and a high boron content, and combinations thereof.

20. (canceled)

21. A component according claim 1 wherein the bearing portion is at least one of at least partially constructed from and at least partially coated with at least one of a material that in use has a magnetic surface, an alloy that is generally non-magnetic but during use becomes magnetic at at least a surface due to strain induced hardening, and an alloy that is generally non-magnetic but that in use develops thin layers of magnetic oxides at a surface due to corrosion.

22-23. (canceled)

24. A component according to claim 1 wherein the magnet assembly comprises at least one permanent magnet.

25. A component according to claim 24 wherein the permanent magnet includes at least one of samarium-cobalt (SmCo) and neodymium-iron-boron (NdFeB).

26. A component according to claim 1 wherein a magnetic field produced by magnet assembly is externally shielded.

27. A component according to claim 1 wherein the passage has an entrance located on a surface which in use is not adjacent to biological tissue and located on or near the bearing surface.

28-29. (canceled)

30. A component according to claim 1 wherein the passage has an approximately circular entrance of from about 0.5 to 5 mm in diameter and has a depth of from about 0.5 to 5 mm.

31. A prosthetic joint suitable for replacing an existing joint comprising two components dimensionally adapted to articulate with each other wherein at least one of the components has a bearing surface including at least one of a magnetic material and a material that in use has a magnetic surface, and wherein at least one of the components comprises:
a portion to secure to a bone;
a bearing portion having a bearing surface wherein at least one reservoir is situated behind the bearing surface;
at least one magnet assembly associated with the reservoir; and
at least one passage extending between a surface of the component and the reservoir.

32. (canceled)

33. A prosthetic joint according to claim 31 wherein one of the components of the joint is made from one of ceramic and a CoCrMo alloy.

34-35. (canceled)

36. A system suitable for replacing a hip joint, which comprises a head and a socket dimensionally adapted to articulate with the head, wherein the head has a bearing surface and a portion for securing the head to a bone, the socket has a bearing surface and a portion for securing the socket to a bone, and at least one of the bearing surface of the socket and the bearing surface of the head substantially comprises one of a magnetic material and a material that in use has a magnetic surface, and wherein at least one of the socket and the head comprises:
at least one reservoir situated behind the bearing surface;
at least one magnet assembly associated with the reservoir; and
at least one passage extending between a surface of the component and the reservoir.

37. (canceled)

38. A system according to claim 36 wherein the head is substantially sphere shaped and the socket is of a corresponding cup shape.

39. A system according to claim 36 wherein the head and the socket are each made at least partially from one of a magnetic material and a material that in use has a magnetic surface, wherein each of the head and the socket may comprise at least one of the same and different materials.

40. A system according to claim 39 wherein a surface of the head and a surface of the socket that articulates with the head both substantially comprise a magnetic material.

41. A system according to claim 40 wherein the head and the socket are both made substantially from non-magnetic material, but contain magnetic material in an area where the head articulates with the socket and an area where the socket articulates with the head.

42. A system according to claim 41 wherein the non-magnetic material for each of the head and the socket is at least one material selected from non-magnetic metals, alloys and ceramics, titanium, titanium alloys, tantalum, CoCrMo, cobalt-nickel-chromium-molybdenum, zirconia, and alumina.

43. A system according to claim 36 wherein the socket comprises at least one of a backing portion and a composite Front section, a bearing section and a backing section for attaching the socket to bone, and an individual bearing section which has an integrated portion for attaching the socket to bone.

44. A system according to claim 36 wherein at least one of the socket and the head has one magnet assembly and one associated reservoir.

45. A system according to claim 44 wherein the magnet assembly and the reservoir are circumferential.

46. A system according to claim 36 wherein the head comprises a core section and at least one outer layer.

47. A system according to claim 46 wherein an outermost layer of the at least one outer layer of the head forms the bearing surface and includes a magnetic material, and the core section comprises a non-magnetic material comprising at least one of titanium, titanium alloy, CoCrMo, CoNiCrMo, stainless steel, and a non-magnetic ceramic.

48. A system according to claim 36 wherein the magnet assembly is located inside one of the socket and the head such that it is coplanar with at least one point of the bearing surface of one of the head and the socket.

49. A system according to claim 48 wherein the magnet assembly is located such that it is coplanar with at least two points of the bearing surface of one of the head and the socket.

50. A system according to claim 36 wherein the magnetic assembly provides suitable field strength and is located such that a magnetic field is produced over the entire area where the head and socket articulate.

51. Use of a prosthetic joint according to claim 31 to reduce the amount of wear debris reaching a prosthesis/bone interface in the prosthetic joint.

52. Use of a prosthetic joint according to claim 31 to reduce or prevent osteolysis.

53. Use of a prosthetic joint according to claim 31 to reduce or prevent aseptic loosening.

54. Use of a prosthetic joint according to claim 31 to reduce a level of circulating and tissue metal ions and wear particles after joint replacement.

55. A component according to claim 2 wherein the portion to secure the component to a bone is a backing portion made from at least one of a non-magnetic metal, a non-magnetic alloy, titanium, a titanium alloy, tantalum, CoCrMo, and cobalt-nickel-chromium-molybdenum.

56. A component according to claim 2 wherein the backing portion has at least one of an outer surface having one of a roughened surface and a coating thereon and one of a roughened and porous coating of at least one of CoCrMo, titanium, titanium alloy, tantalum, and hydroxyapatite.

57. A component according to claim 2 wherein the bearing portion is part of a composite front section comprising:
a non-magnetic shell that contains the reservoir and the magnet assembly associated with the reservoir;
the bearing portion, wherein the bearing portion is located on the inner surface of the shell; and
an additional non-magnetic layer on an outer surface of the shell.

58. A component according to claim 2 wherein the bearing portion is at least one of an individual section attachable directly to the portion to secure the component to a bone, an individual section integrated with the portion to secure the component to a bone, one of wholly and partially constructed from a magnetic material, and one of wholly and partially coated with a magnetic material.

59. A component according to claim 58 wherein an outer surface of the bearing portion is coated with a material that encloses the magnet assembly within the component to prevent dislodgement of the magnet assembly during at least one of insertion and use.

60. A component according to claim 58 wherein the magnetic material is at least one of a magnetically soft material with low coercivity and a magnetic material having a high saturation magnetic flux density and high relative permeability such that wear debris created in use around the joint component is pulled towards the magnet assembly.

61. A component according to claim 58 wherein the magnetic material is selected from a group consisting of metal matrix composites, metal alloys, a metal alloy selected from cobalt based alloys, nickel based alloys, magnetic steels, and iron based alloys, metal oxides, a metal oxide selected from oxides of nickel, iron, cobalt, and manganese, intermetallic compounds, ceramics, a ceramic selected from oxides and nitrides of nickel, iron, cobalt, and manganese, amorphous metal alloys, an amorphous metal alloy selected from cobalt, nickel, and iron based amorphous alloys with at least one of a high silicon and a high boron content, and combinations thereof.

62. A component according to claim 2 wherein the bearing portion is at least one of at least partially constructed from and at least partially coated with at least one of a material that in use has a magnetic surface, an alloy that is generally non-magnetic but during use becomes magnetic at at least a surface due to strain induced hardening, and an alloy that is generally non-magnetic but that in use develops thin layers of magnetic oxides at a surface due to corrosion.

63. A component according to claim 2 wherein the magnet assembly comprises at least one permanent magnet.

64. A component according to claim 63 wherein the permanent magnet includes at least one of samarium-cobalt (SmCo) and neodymium-iron-boron (NdFeB).

65. A component according to claim 2 wherein a magnetic field produced by the magnet assembly is externally shielded.

66. A component according to claim 2 wherein the passage has an entrance located on a surface which in use is not adjacent to biological tissue and located on or near the bearing surface.

67. A component according to claim 2 wherein the passage has an approximately circular entrance of from about 0.5 to 5 mm in diameter and has a depth of from about 0.5 to 5 mm.
